(19) United States
(12) Patent Application Publication
(10) Pub. No.: US 2021/0278320 A1
(43) Pub. Date: Sep. 9, 2021

(54) Decontamination Assessment

(71) Applicant: Robert Laughlin, Miramar, FL (US)

(72) Inventor: Robert Laughlin, Miramar, FL (US)

(21) Appl. No.: 16/810,661

(22) Filed: Mar. 5, 2020

(51) Int. Cl. G01N 1/04; G01N 33/36

(52) U.S. Cl. CPC G01N 1/04; G01N 33/36

(57) ABSTRACT

A device and method of use for assessing the state of decontamination or contamination of the surface of an article. The device is comprised of a handle and a collector assembly. The collector assembly has a collector surface that is rubbed onto the surface being tested thereby capturing a sample of contaminants on that surface. The collector assembly is then hermetically sealed in a bag and returned to the testing laboratory for analysis.

### CROSS-REFERENCES TO RELATED APPLICATIONS

None.

### STATEMENT REGARDING FEDERAL SPONSORED RESEARCH OR DEVELOPMENT

None.

### NAMES OF PARTIES TO A JOINT RESEARCH AGREEMENT

None.

### REFERENCE TO A “SEQUENCE LISTING”, A TABLE, OR A COMPUTER PROGRAM LISTING APPENDIX SUBMITTED ON COMPACT DISC AND INCORPORATION-BY-REFERENCE OF THE MATERIAL ON THE COMPACT DISCLOSURE

None.

### STATEMENT REGARDING PRIOR DISCLOSURES BY AN INVENTOR OR JOINT INVENTOR

None.

### BACKGROUND OF THE INVENTION

### 1. Field Of The Invention

The present invention relates to assessing the state of decontamination remediation of clothing, equipment and other objects, and more particularly, to an improved device and method of use to collect a surface sample from, for example first responder equipment, in the process of chemical testing for residual contaminants and foreign objects.

### 2. Description Of Related Art Including Information Disclosed Under 37 Cfr 1.97 And 37 Cfr 1.98

Several designs for chemical testing have been designed in the past. None of them, however, includes features and methods that are adapted for use by first responders in the field to sample and assess contaminant remediation measures.

Specifically, the present device includes a handle assembly allowing a user to grasp a collector assembly without touching the collector assembly. The collector assembly is then rubbed in contact with the surface to be tested. The collector assembly further includes a collection enhancing surface to obtain a sample from the surface of the subject object. A sterile container is also provided to individually sequester the collector assembly in a hermetically sealed environment for delivery to a testing laboratory.

The constituent elements of the device and the method used or obtained a sample are adapted to efficient use by an individual not particularly skilled in chemical analysis or technical sample collection. The simple tools provided in a unified kit, along with straightforward instructions, allow nearly anyone to obtain a chemical sample of the surface to be tested for decontamination assessment.

Other patents describing the closest subject matter provide for a number of more or less complicated features that fail to solve the problem in an efficient and economical way. None of these patents suggest the novel features of the present invention.

A brief abstract of the technical disclosure in the specification and title are provided as well for the purposes of complying with 37 CFR 1.72 and are not intended to be used for interpreting or limiting the scope of the claims.

Without limiting the scope of the invention, a brief summary of some of the claimed embodiments of the invention is set forth below. Additional details of the summarized embodiments of the invention and/or additional embodiments of the invention may be found in the detailed description of the invention below.

### BRIEF SUMMARY OF THE INVENTION

It is one of the main object of the present invention to provide a device to easily assess an article after decontamination.

It is another object of this invention to provide a device that is self-contained and readily used by someone with minimal technical training.

It is another object of the present invention to provide a device and method of use to improve safety of first responders after decontaminating equipment by ensuring remediation.

It is yet another object of this invention to provide such a device and method of use that is inexpensive to manufacture and maintain while retaining its effectiveness.

Further objects of the invention will be brought out in the following part of the specification, wherein detailed description is for the purpose of fully disclosing the invention without placing limitations thereon.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE SEVERAL VIEWS OF THE DRAWING

With the above and other related objects in view, the invention exists in the details of construction and combination of parts as will be more fully understood from the following description, when read in conjunction with the accompanying drawings in which:

FIG. 1 shows a prospective view of a handle assembly connected to a collector assembly.

FIG. 2 shows a prospective view of an example of a collector assembly.

FIG. 3 shows an example of a collector assembly.

FIG. 4 shows an example of a collector assembly.

FIG. 5 shows an example of a collector assembly.

FIG. 6 shows an example of the device in use testing a fireman's uniform.

FIG. 7 shows additional elements in a unified process to ensure quality of equipment and health risk issues used after potential exposure to harmful substances.

### DETAILED DESCRIPTION OF THE INVENTION

While this invention may be embodied in many different forms, there are described in detail herein specific embodiments of the invention. This description is exemplary of the principles of the invention and is not intended to limit the invention to the particular embodiments illustrated and described.

For the purpose of this disclosure, like reference numerals in the figures shall refer to like features unless otherwise indicated or is obvious by context.

The subject device and method of use is sometimes referred to as the device, the invention, the decontamination assessment device or tool, the tester, the testing device, the collector device, the machine or other similar terms. These terms may be used interchangeably as context requires and from use the intent becomes apparent. The masculine can sometimes refer to the feminine and neuter and vice versa. The plural may include the singular and singular the plural as appropriate from a fair and reasonable interpretation in the situation.

Referring now to the drawings, where the present invention is generally referred to with numeral 10, it can be observed that it basically includes a handle assembly 12, a grip 14, a release 16, a tab 18, a collector assembly 20, a body 22, a collector 24, a slot 26, a mesh 28, a bristled 30, a pad 32, a set of gear 34, a stain 36, a bag 38, an air sampler kit 40, and a box 42.

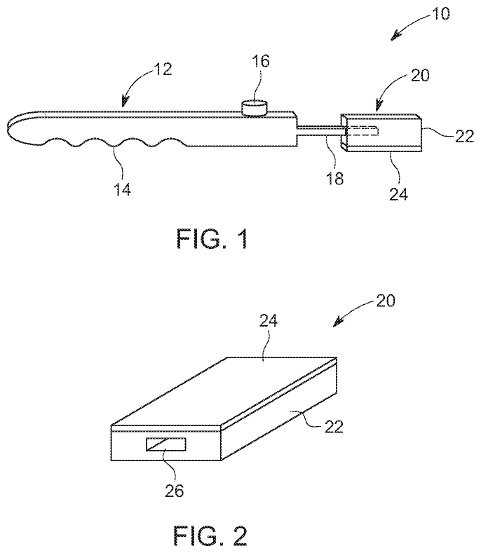

Looking at FIG. 1, an example of the assembled decontamination assessment tool is shown. Generally, the decontamination assessment tool is comprised of a handle assembly 12 connected to a collector assembly 20. The handle assembly 12 is dimensioned and adapted to fit a typical human hand around the grip 14. In typical use, the user grasps the handle assembly 12 around the grip 14 leaving the tab 18 protruding from the device.

Prior to using the decontamination assessment tool a sterile collector assembly 20 is fitted over the tab 18. The tab 18 may be friction fit into the slot 26 formed into the body 22 of the collector assembly 20. The interior of the slot 26 is dimensioned to fit tight enough over the tab 18 to prevent it from disconnecting from the tab 18 during the normal surface sampling, yet be able to readily be detached for placement inside a bag 38 for returning to the testing facility.

The tab 18 may also include a retractable barb or similar element at or near the tip of the tab 18 that is retracted by the release 16 on the handle assembly 12. By pressing the release 16 on the handle assembly 12 the barb may be retracted and the collector assembly 20 will easily slide off the tip of the tab 18.

The tab 18 and slot 26 combination is intended to include other similar means to connect a handle assembly 12 to the collector assembly 20. For example, instead of a tab 18 the handle assembly 12 may include a clip, magnet or other means to removably connect the collector assembly 20 to the handle assembly 12.

Examples of collector assemblies 20 are shown in FIGS. 2, 3, 4 and 5. Each collector assembly 20 is comprised of a body 22 having a collector 24 exposed on an exterior surface. A slot 26 is formed into the body 22 for connection to the handle assembly 12.

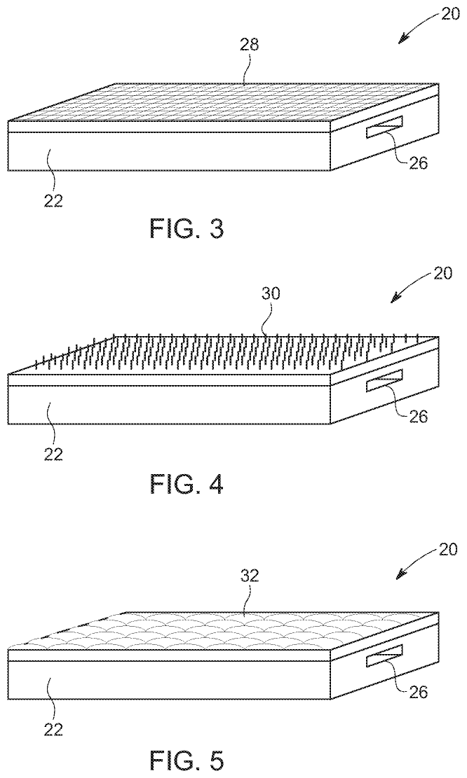

FIG. 3 shows a version of a collector assembly 20 where the collector 24 is embodied in a mesh 28. The mesh 28 may be a woven or non-woven, alloy or polymer, that is adhered to an exterior surface of the body 22. By nature of being a mesh 28, any contaminants remaining on a tested surface will adhere to the mesh 28 when robbed onto that surface. The mesh 28 will have the ability to both scrape and agitate the surface being tested dislodging particles and contaminants that remain adhered to the mesh 28.

The mesh 28 may appear to be a fine screen or layer of entwined filaments. The mesh 28 may be on a single side of the body 22 or may cover multiple exterior surfaces of the body 22. The mesh 28 is generally permanently bonded to the body 22. The entire collector assembly 22 including the mesh 28 is returned to the testing facility for analysis for the effectiveness of the subject decontamination procedure.

FIG. 4 shows an alternate version of a collector assembly 20. In this version, the collector 24 is embodied as a series of bristles 30. Like other versions of the collector assembly 20, the bristles 30 may be made of a metallic or polymer material embedded into the collector 24. The bristles 30 protrude a short distance, generally from about a ½ mm to 5 mm in length. Different length of Bristol 30 may be suited to particular types of contaminants and surfaces being tested.

FIG. 5 shows yet another embodiment of a collector assembly 20. The collector surface is characterized as a pad 32. The collector assembly 20 has a body 22 and slot 26 similar in form and function to other versions of the collector assembly 20. The pad 32 provides and another type of surface that when rubbed on a substrate being tested will pick up and hold materials such as particles, fibers, smoke, oil, waxes and other types of contaminants that are being tested.

The pad 30 is adhered to the body 22 of the collector assembly 20. The pad 30 may be fabricated of materials such as a synthetic sponge, fabrics (both woven and nonwoven) or other fibrous or porous materials that can adhere contaminants when robbed upon a substrate. The pad 30 may also have a static charge to attract and retain particulate contamination.

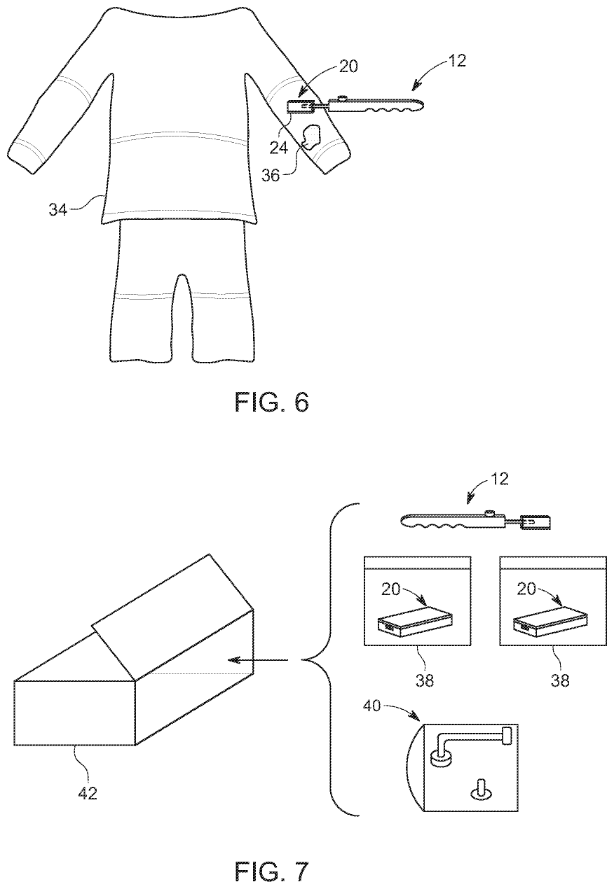

FIG. 6 shows a set of gear 34 embodied in a set of fireman's protective jacket and pants. In this example, the set of gear 34 has a visible stain 36 on the sleeve. An operator of the device grasps the handle assembly 12 with a collector assembly 20 firmly connected. The operator then rubs the collector 24 side of the collector assembly 20 against the sleeve of the set of gear 34.

As the collector 24 scrapes against the set of gear 34 any particulate contaminant will be dislodged from the set of gear 34 and become attached to the collector 24. The collector 24 is not designed to clean the surface by removing all particulate contaminant but instead is to take a sample of any contaminant on the substrate for trace analysis.

Although the set of gear 34 in FIG. 6 shows a stain 36, any part of the set of gear 34 may be tested by rubbing the collector 24 against the set of gear. The stain 36 may be of particular interest to the person using the decontamination assessment tool. However, the decontamination assessment tool is not in any way limited to visible stain's 36 only.

A user of the decontamination assessment tool may take a sample of the stain 36 in particular by rubbing the collector 24 against the stain alone. Then, that particular collector 24 is hermetically sealed inside a bag 38 so that cross-contamination with other collector assemblies 20 or other contaminant sources are avoided. The person testing may then sample additional areas of the set of gear 34 away from the stain 36 to collecting other sample on the collector 24 of any contaminants on the surface of the set of gear 34.

Conducting a decontamination assessment with the device may be done after remediation efforts to decontaminate a particular substrate is performed to ensure the sufficiency of decontamination efforts. For example, after a firefighter uses the set of gear 34 in the course of their role as a first responder the gear 34 should routinely be cleaned and maintain. Then, after standard of decontamination has been completed a decontamination assessment with the present device may be conducted to ensure that the decontamination was complete and there is no risk posed to the firefighter when using the set of gear 34 again.

Alternatively, the decontamination assessment procedure can be used prior to any cleaning or other remediation efforts. This may establish a baseline of contamination levels. This may be important to determine the exposure level that a particular first responder, such as a firefighter, may have encountered in the line of duty.

It should be appreciated that the handle assembly 12 and collector assembly 20 shown in the drawings and described herein are mere examples of the physical form of these parts. Other shapes and configurations of the various elements with similar performance characteristics may be equally substituted for those shown. For example, the collector assembly 20 may be different shapes such as cylindrical or other shape. Also equally functional is a design where the handle assembly 12 in collector assembly 20 do not readily separate but are unified into one assembly.

An important aspect of the present device is that it is readily usable by an end-user with minimal training or technical skill. The decontamination assessment kit is delivered to the end-user complete with a handle assembly 12, one or more collector assemblies 20, sufficient bags 38 to hermetically seal the collector assemblies 20 after gathering the samples, and optional air sampler kit 40 and a return shipping box.

The air sampler kit 40 is a separate device used to collect air samples for contamination source and is not functionally directly related to the decontamination assessment device. However the air sampler kit 40 is often provided to complete the process of decontamination assessment of both surfaces being tested with the present device and air quality from breathing equipment or ambient air with the air sampler kit 40.

The collector assemblies 20 are provided in sealed bags to ensure that they are contaminant free prior to their use. The new collector assemblies are individually provided in the sealed bag 38 that may include a tamper evident seal. For example, a portion of the bag opening may include a seal or other feature that is torn or broken when opening a collector assembly 20 for the first time.

The bag 38 is also provided on new, clean collector assemblies 20 so that upon opening a new collector assembly 20 the operator can insert the tab 18 into the slot 26 of the collector assembly 20 without having to touch the collector assembly 20 at all. This ensures that the collector 20 is not contaminated from touching or contact with other services prior to being rubbed on the specific substrate intended to be tested. The clean collector assemblies 20 could equally be provided in a sealed box or other container as an alternative to a bag.

As shown in FIG. 7, after air samples and surface samples have been collected they are placed into a return shipping box 42 along with the reusable handle assembly 12. The box is uniquely identified with tracking information including information to derived the customer or origin of the samples contained within the box 42 the box is delivered to a remote testing laboratory where detailed chemical analysis is conducted on both the collector assemblies 20 and, if provided, the air sampler kit 40.

Once analyzed results can be delivered to the customer electronically. It a replacement kit with fresh, clean collector assemblies 20 and a new air sampler kit 40 can be provided to the customer. Additional samples of potentially contaminated equipment and air can then be tested again by the end-user, returned via post to the analyzing laboratory and results provided to the end-user.

An important version of the invention can be fairly described as a decontamination assessment device comprised of a handle assembly and a collector assembly. The collector assembly is affixed to an end of the handle assembly. The collector assembly has a collection surface that when rubbed onto a substrate collects a surface contaminant from the substrate. The collector assembly is hermetically sealed in a container and delivered to a testing facility to determine if the substrate has been decontaminated.

The invention can also be fairly described as a process for decontamination assessment comprised of the steps of first providing a substrate (such as clothing, furniture, upholstery or other solid material) that has undergone a decontamination procedure. For example, after a fire rescue mission. Then, providing a handle assembly with a grip on a first end and a connector on a second end. The user grabs the grip to hold the device. Then providing a new collector assembly, clean and sealed inside a first container. The collector assembly has an integral collection surface, such as those noted in reference to FIGS. 3-5. Then, so as to not contaminate the collection surface with the users hands or other things it could touch, unsealing the container and grasping the grip and affixing the collector assembly to the connector on the handle assembly without the collection surface being contaminated by a non-intended-surface. Essentially connecting the collector assembly to the handle assembly while the collector assembly is still in the sterile bag. Then grasping the grip and rubbing the collection surface on the substrate to collect a sample of any surface contaminant, if present. Then, inserting the collector assembly into a second sterile container. Then, separating the handle assembly from the collector assembly wherein the collector assembly remains inside the second container for transport. Hermetically sealing the second container with the collector assembly inside. Then, sending the collector assembly sealed inside the second container to a testing facility. Then, receiving a result from an analysis of the collection surface performed by the testing facility. And finally, determining whether the substrate has been sufficiently decontaminated by the decontamination procedure and is therefore suitable for continued use. If not then the substrate being tested may be re-decontaminated or discarded.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

I/we claim:

1. A decontamination assessment device comprised of a handle assembly and a collector assembly;
the collector assembly is affixed to an end of the handle assembly;
the collector assembly has a collection surface that when rubbed onto a substrate collects a surface contaminant from the substrate;
the collector assembly is hermetically sealed in a container and delivered to a testing facility to determine if the substrate has been decontaminated.

2. A process for decontamination assessment comprised of the steps of:
providing a substrate that has undergone a decontamination procedure;
providing a handle assembly with a grip on a first end and a connector on a second end;
providing a collector assembly, clean and sealed inside a first container;
wherein the collector assembly has an integral collection surface;
unsealing the container;
grasping the grip and affixing the collector assembly to the connector on the handle assembly without the collection surface being contaminated by a non-intended-surface;
grasping the grip and rubbing the collection surface on the substrate;
inserting the collector assembly into a second container;
separating the handle assembly from the collector assembly wherein the collector assembly remains inside the second container;
hermetically sealing the second container with the collector assembly inside;
sending the collector assembly sealed inside the second container to a testing facility;
receiving a result from an analysis of the collection surface performed by the testing facility;
determining whether the substrate has been sufficiently decontaminated by the decontamination procedure and is therefore suitable for continued use.
